(19) United States
(12) Patent Application Publication
(10) Pub. No.: US 2004/0152096 A1
(43) Pub. Date: Aug. 5, 2004

(54) Surf2lead

(76) Inventors: Axel Dietrich, Berlin (DE); Ola Engkvist, Berlin (DE); Ulrich Rester, Berlin (DE); Li-hsing Wang, Berlin (DE); Paul Wrede, Berlin (DE)

Correspondence Address: SUTHERLAND ASBILL & BRENNAN LLP, 999 PEACHTREE STREET, N.E., ATLANTA, GA, 30309, US

(21) Appl. No.: 10/477,679

(22) PCT Filed: May 10, 2002
(86) PCT No.: PCT/EP02/05180

(51) Int. Cl. C12Q 1/68; G01N 33/53; G01N 33/543

(52) U.S. Cl. 435/6; 435/7.1; 436/518

(57) ABSTRACT

The present invention relates to a method for generating a focussed compound library containing ligand compounds being capable of binding to a pre-selected target. In particular, the invention relates to an integrated lead identification approach starting out from the three-dimensional structure of a selected target.

The present invention relates to a method for generating a focussed compound library containing ligand compounds being capable of binding to a preselected target or receptor. In particular, the invention relates to an integrated lead identification approach starting out from the three-dimensional structure of a selected target or receptor.

One major bottleneck of current drug discovery and drug development is the search for lead structures. To find new lead structures great efforts are made on the development of fast screening tools. Currently combinatorial chemistry, high throughput screening (HTS) and laborious medicinal chemistry are the major columns in drug discovery. These technologies result in an effective search of lead structures. Anyway this process is still very expensive and time-consuming. Therefore, computer-based algorithms will be significant improvement in minimizing costs and time.

It is therefore an objective of the present invention to provide a method for further improving and accelerating the virtual screening of databases for specific ligand compounds.

It is a further objective of the present invention to provide a method for generating a focussed compound library comprising ligand compounds being capable of binding to a preselected target.

It is another objective of the present invention to provide an improved method for generating pharmacophores.

The objectives of the present invention are solved by a method for generating a focussed compound library comprising the steps

(a) selecting a target,

(b) providing information about the three-dimensional structure of the target,

(c) identifying interaction points in the active site of the target from the three-dimensional structure,

(d) generating an inverse active site consisting of ligand atom positions expressed as interaction types,

(e) generating two or more shells of inverse active sites having different distances to the target for each interaction type,

(f) extracting a pharmacophore for a ligand by clustering interaction points of the inverse active sites on the shells,

(g) employing the inverse active site pharmacophore as query for the identification of suitable ligand compounds, and

(h) collecting the suitable ligand compounds identified so as to form a focussed library.

The method of the present invention, also called Surf2Lead®, is an integrated strategy for fast lead identification. The basic concept is the discovery of pharmaceutical lead compounds starting out from the three-dimensional target or receptor structure. In combination with computational screening approaches (e.g. PHACIR®) new biologically active ligands can be found.

The present invention, called Surf2Lead®, relates to a novel lead detection strategy. Surf2Lead® identifies ligands capable of specifically binding given targets of protein domains based on the 3D information of the target.

The Surf2Lead® strategy comprises the following steps:

1. Identification of possible interaction points in the active site of a given target. Any 3D target structure (X-ray, NMR, theoretical model) or any 3D active site information can be used.

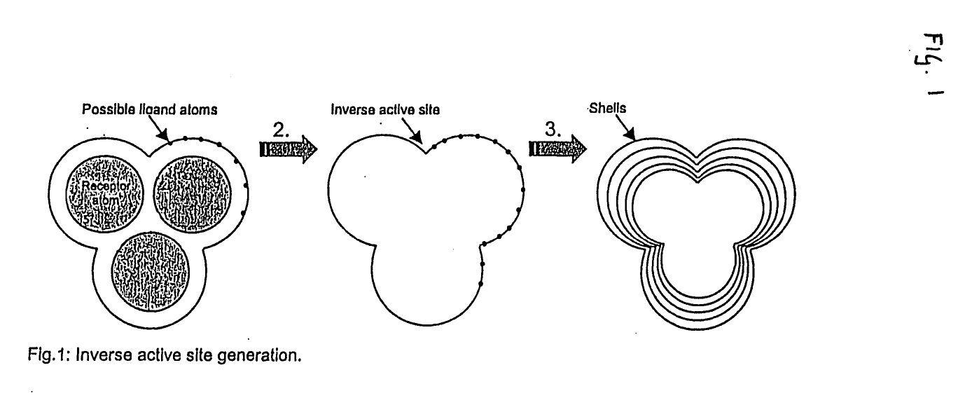

2. Generation of an “inverse active site” to define possible ligand atom positions in the active site. Atoms are expressed as interaction types describing their chemical properties (FIG. 1). The distance to the target atoms depends on the interaction type.

3. Several shells of inverse active sites with different distance to the target are generated for each interaction type. The density of possible ligand atoms on these shells depend on the distance to the target.

4. The shells are used to extract a possible pharmacophore describing possible ligands by clustering the interaction points. The clustering depends on a given radius around each dot which is examined and on the density of the dots on the shells. Optionally, the distance to the receptor can be taken into account. And optionally

5. Lead compound identification: The inverse active site pharmacophore serves as a query for computer-based lead identification tools (e.g. PHACIR®). The resulting focussed library of potential leads has to be tested in vitro.

According to the method of the invention, first, a desired target is selected, e.g. a target associated with a particular disease. Information about suitable targets can be obtained, for example, by previous testing or from literature.

Next, the three-dimensional structure of the target is determined according to the invention. Thereby either the three-dimensional structure of the whole target or only the three-dimensional structure of parts can be determined. In particular, the three-dimensional information about active sites or the three-dimensional structure of target pockets is sufficient in many cases. The three-dimensional structure actually can be determined, e.g. by X-ray structure analysis, NMR or other methods, or estimated in a theoretical model.

Next, interaction points in the active site of the target are identified from the three-dimensional structure. Each target atom will be analysed according to their pharmacophoric properties (e.g. hydrogen bond acceptor, lipophilic, etc.). These properties are defined within a table describing different functional groups and their pharmacophoric pattern. The resulting pharmacophoric properties of the target atoms serve as the base for the concurrent generation of the inverse active site.

Next, an inverse active site is generated consisting of ligand atom positions expressed as interaction types. The interaction types describe the chemical properties of the ligand atoms, whereby the distance to the target atoms depends on the interaction type. Examples of interaction types are hydrogen bond donor, hydrogen bond acceptor, lipophilic, aromatic, positive or negative. To obtain an inverse active site, each target atom comprising a pharmacophoric property is examined. At first, a sphere is generated around this target atom with a given radius depending on the interaction type. This surface of this sphere is filled with dots. The dots obtain the “inverse” pharmacophoric property of the target atom, e.g. hydrogen bond acceptor target atom is surrounded by hydrogen bond donor dots. These dots represent potential ligand atom positions (PLAP). To examine only the surface dots all inner dots within the target structure will be deleted. By executing this procedure a surface shell for each interaction type can be obtained.

In the method of the invention two or more shells of inverse active sites having different distances to the target are generated for each interaction type. More preferably, 3 to 20, in particular, 4 to 10 shells are formed. The radii of the shells normally range within empirically determined values for the distinct interaction types, e.g. lipophilic from 2.5 to 4.5 Å, but can vary in a wide range. The distances of the dots on each shell depend on the distance to the target atom and will be higher for the outer and inner shells and smaller for middle shells to attain a stronger weight of the medium distances of interactions. The dot distance can vary in a wide range of 0.01 up to 10 Å.

Finally, a pharmacophore is extracted by clustering the interaction points of the active inverse sites of the shells. The obtained PLAP shells serve as the base for extracting pharmacophoric information by means of clustering. This is effected by determining the density of the PLAPs in space. Regions with higher density are separated from regions with lower density. From each cluster pivotal points are selected, representing pharmacophoric points. The number of representative pharmacophoric points is flexible and depends on the preset density and radii.

Providing a pharmacophore as described above is one aspect of the invention.

The thus obtained pharmacophore serves as query, e.g. for computer-based lead identification tools for the identification of suitable ligand or lead compounds. In particular, small molecules having a molecular weight of between 100 and 2,000 Dalton, in particular, between 150 and 1,000 Dalton, which are no peptides, are preferred lead structures.

The identified lead compounds can be collected or combined in a library. It is also possible to generate a focussed library of potential lead candidates by means of a limit value, starting out from a starting compound library. The lead candidates can then be tested for their activity in vitro.

The invention particularly includes the enrichment of biologically active compounds in a focussed substance library which is obtained from given starting databases. The pharmacophores generated according to the invention can be used for the extraction of ligand compounds from a substance library or for focussing a database according to specific similarity criteria.

The method according to the invention, in particular, can be employed for the development of lead structures, drugs or biologically active compounds. It is particularly suitable for applications in human or veterinary medicine and in plant protection.

The invention is further described by the following Examples and the Figures, wherein

FIG. 1 shows an inverse active site generation

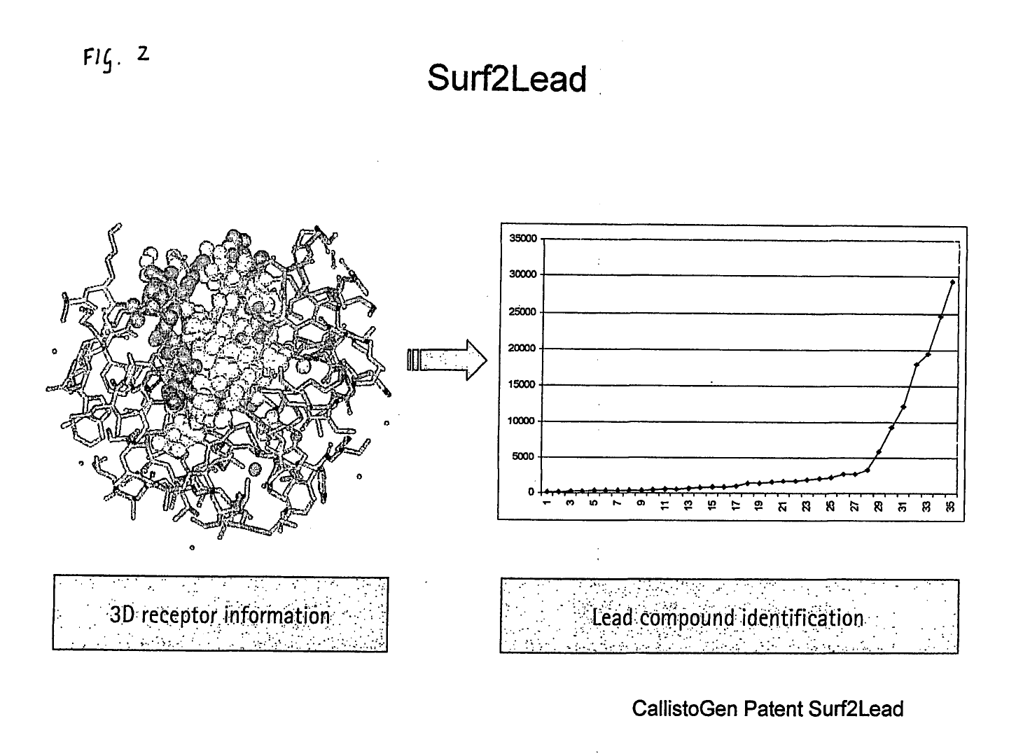

FIG. 2 shows the concept of lead compound identification starting out from 3D target information, and

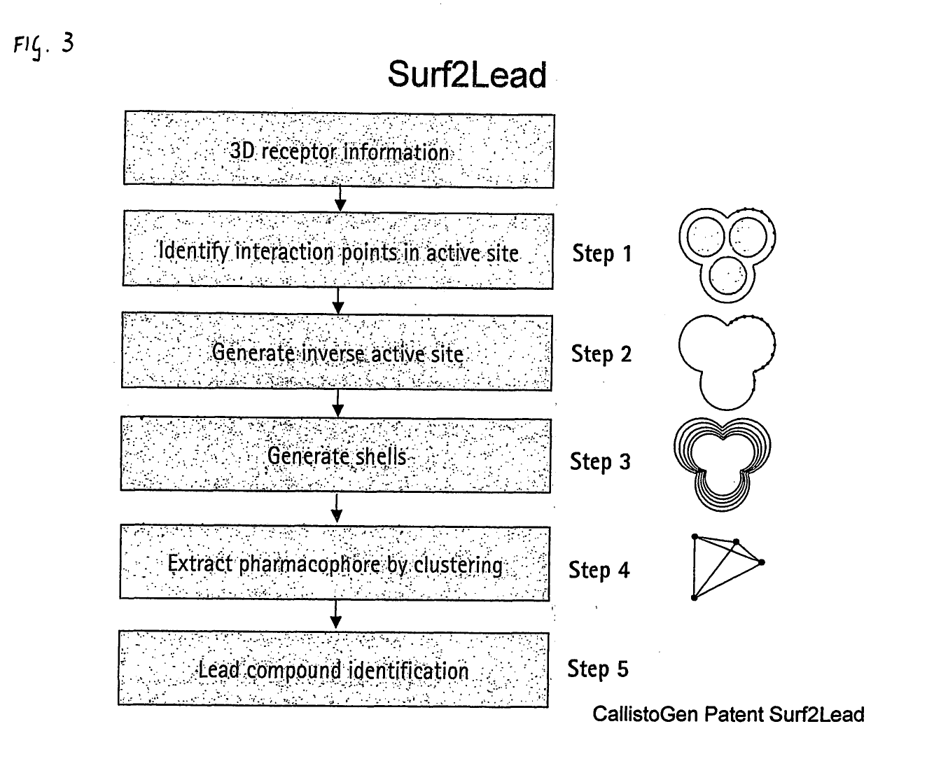

FIG. 3 shows an overview of the Surf2Lead® integrated lead identification approach.

1. A method for generating a focussed compound library comprising the steps
(a) selecting a target,
(b) providing information about the three-dimensional structure of the target,
(c) identifying interaction points in the active site of the target from the three-dimensional structure,
(d) generating an inverse active site consisting of ligand atom positions expressed as interaction types,
(e) generating two or more shells of inverse active sites having different distances to the target for each interaction type,
(f) extracting a pharmacophore for a ligand by clustering interaction points of the inverse active sites of the shells,
(g) employing the inverse active site pharmacophore as query for the identification of suitable ligand compounds, and
(h) collecting the suitable ligand compounds so as to form a focussed library.

2. A method for generating a pharmacophore comprising the steps:
(a) selecting a target,
(b) providing information about the three-dimensional structure of the target,
(c) identifying interaction points in the active site of the target from the three-dimensional structure,
(d) generating an inverse active site consisting of ligand atom positions expressed as interaction types,
(e) generating two or more shells of inverse active sites having different distances to the target for each interaction type,
(f) extracting a pharmacophore for a ligand by clustering interaction points of the active inverse sites of the shells.

3. The method according to claim 1 or 2, wherein a two-point pharmacophore, a three-point pharmacophore or a four-point pharmacophore is generated.

4. The method according to any of the preceding claims, wherein a target is selected which is associated with a particular disease.

5. The method according to any of the preceding claims, wherein the three-dimensional structure of the whole target or the three-dimensional structure of parts of the target is provided.

6. The method according to claim 5, wherein the three-dimensional structure is obtained by a theoretical model or/and experiments.

7. The method according to any of the preceding claims, wherein interaction points are identified by assigning pharmacophoric properties to the target atoms.

8. The method according to any of the preceding claims, wherein an inverse active site is generated by spheres around interaction points covered with dots representing potential ligand atom positions.

9. The method according to any of the preceding claims, wherein 3 to 20 shells of inverse active sites are generated.
